(19) United States
(12) Patent Application Publication
(10) Pub. No.: US 2013/0064854 A1
(43) Pub. Date: Mar. 14, 2013

(54) METHODS OF TREATING ALLERGIES WITH SUBSTANTIALLY PHENOL-FREE CARRIERS

(71) Applicants: Robert W. Pomrenke, Jacksonville, FL (US); William Goldberg, Winnetka, IL (US); Patrick D. McGrath, Gurnee, IL (US)

(72) Inventors: Robert W. Pomrenke, Jacksonville, FL (US); William Goldberg, Winnetka, IL (US); Patrick D. McGrath, Gurnee, IL (US)

(73) Assignee: Antigen Laboratories, Inc., Liberty, MO (US)

(21) Appl. No.: 13/607,221

(22) Filed: Sep. 7, 2012

Related U.S. Application Data
(63) Continuation-in-part of application No. PCT/US2011/027853, filed on Mar. 10, 2011.
(60) Provisional application No. 61/313,438, filed on Mar. 12, 2010.

(51) Int. Cl. A61K 39/35; A61P 37/08; A61P 37/02; A61K 39/36

(52) U.S. Cl. 424/275.1

(57) ABSTRACT

A method for treating a patient suffering from allergy by desensitizing the patient to an allergen component that includes orally, sublingually, or intranasally administering to the patient a therapeutically effective amount of an immunomodulating composition and successively repeating the administration at a selected interval with the therapeutically effective amount of each successive administration comprising an increasing amount of the allergen component. The composition includes an amount of the allergen component that is sufficient to impart an immune response and a pharmaceutically acceptable carrier that is at least substantially free of phenol. The successive repeating occurs a sufficient number of times to reduce patient sensitivity to the allergen component, and avoids or minimizes oral adverse effects. An immunomodulating product is also described.

### CROSS-REFERENCE TO RELATED APPLICATION

This application is a continuation-in-part application of PCT/US2011/027853, filed Mar. 10, 2011, which claims the benefit of U.S. Provisional Patent Application No. 61/313,438, filed Mar. 12, 2010, the contents of each of which is hereby incorporated herein in its entirety by express reference thereto.

### TECHNICAL FIELD

The present invention relates generally to methods of treating allergies by administering compositions comprising an allergen component and a carrier that is at least substantially free of phenol. Administration of the compositions causes a minimum of adverse effects. The present invention also relates to immune modulating products that include an allergen component and a carrier that is at least substantially free of phenol associated with dosing instructions.

### BACKGROUND OF INVENTION

It has long been known that many persons show allergic reactions upon contact with certain naturally occurring materials, such as various plants and animals. Some such allergenically active agents include pollens, epithelia, especially animal hairs, dusts, especially house dust, insects, and fungi.

Over 10 million people just in the United States are known to be allergic to animals. Household pets are the most common source of such reactions. Many people think the hair of cats and dogs provokes pet allergies. The major allergens, however, are proteins secreted by oil glands in the animal's skin and shed in dander; and in the saliva, which sticks to the hair when the animal licks itself. When the saliva carrying the proteins dries, the proteins float into the air and are inhaled by people. Some rodents, such as guinea pigs and gerbils, have become increasingly popular as household pets. They, too, can cause allergic reactions in some people, as can mice and rats.

Between 6 and 7 million U.S. persons are reportedly affected by food allergies. Food allergies are different from food intolerances, since food intolerances do not involve the immune system. Up to 3 million Americans are highly allergic to peanuts and tree nuts. Eight foods account for 90 percent of food allergies: milk, fish, peanuts, tree nuts, eggs, soy, wheat, and shellfish. In these cases, systemic reactions may be severe, such as onset of allergic shock. To avoid serious consequences including death, people allergic to foods carry injectable epinephrine. Even so, treatment or preventative measures for food allergies are often only marginally effective. The primary therapy is simply total avoidance of the specific allergen. Conventional subcutaneous allergy shots are typically ineffective against food allergies.

Approximately 5 million people in the Unites States are reportedly allergic to bee or wasp stings, in many cases with potentially life threatening symptoms. Three out of 5 of allergic people stung will experience a more severe reaction if stung again.

The etiology and root causes of the multitude of individual diseases or conditions included within the generic definition of allergy are not completely elucidated or known at this time. The generic condition known in medicine as “allergy” connotes an altered reaction of tissues or other systemic parameters in certain persons on exposure to certain agents which, in similar amounts are innocuous to other persons. These altered reactions of the allergic person may be of various types, ranging from a more or less intense skin sensitivity to a severe and sometimes fatal systemic shock reaction.

At the present time, it is the consensus of the medical world that the allergic reactions suffered by all susceptible persons are mediated, if not basically caused, by various exciting agents, commonly referred to as “allergens” or “antigens.” The presence of these allergens cause, in those individuals who are sensitive to the specific allergens involved, the production of specific antibodies. The interaction of antibodies on the surface of certain effector cells, such as mast cells, with antigen causes the degranulation of the mast cell and the release of a number of pre-formed mediators. The most significant of these mediators is believed to be histamine, which is responsible for the common symptoms of allergy, such as runny nose, itchy and watery eyes, urticaria and exacerbation of asthma symptoms.

A person who is allergic may be sensitive to only one allergen, but multiple sensitivities tend to be more common. Various types of allergens are, for example, pollens, fungi, vegetable or animal epithelia, cosmetics, and house dust components; foods, for example, eggs, shellfish, nuts, and strawberries; infectious agents, for example, bacteria, fungi, molds; dust mites, and contactants, for example, plants, flowers, chemicals, furs, and cosmetics. These allergic materials may contain one or more allergens to which a person is sensitive and exposure to these allergic materials will, in the susceptible individual, cause an allergic attack, manifested by one or more allergic symptoms, the severity of which depends upon the degree of exposure, as well as the nature of the allergic material to which the individual is exposed.

It is often important to therapy to determine the types of allergic materials and/or allergens to which the allergic person is susceptible. This determination is required to know which allergic materials the person should be isolated from, as well as to ascertain the type of treatment required to prevent, ameliorate or therapeutically treat the allergy. The diagnostic procedures that have been developed to date include one that is most commonly employed to determine the identity of the allergens to which the patient is sensitive, the skin test. In the skin test diagnostic procedure, a small amount of an allergen extract is administered cutaneously and the local allergic reaction, i.e., wheal and flare, observed to determine the person's sensitivity to the test allergen.

Once the identity of the allergen(s) to which the person is sensitive has been determined, the type of therapeutic treatment required to prevent future allergic attacks can be selected. Where the total elimination of the allergen from the presence of the patient is not feasible, for example, where the allergen is a pollen or house dust component, one of the prime methods of treatment is that of desensitization or hyposensitization. This method of treatment involves the successive injecting of an extract of the causative allergen in a series of gradually increasing doses until the patient slowly builds up an immunity threshold, which will permit exposure to a normal concentration of the allergen without an allergic attack.

Clinical experience has shown that allergic reactions can be lessened by subcutaneous injections of extracts of the substances causing the allergy. Heretofore, aqueous liquids, including phenolated physiologic NaCl, 50% glycerin, and others with or without added human serum albumin have been employed for the production of such allergenic extracts or preparations in general, sometimes after previously degreasing the allergen-containing starting material with ether.

Generally, the thus-obtained solutions, after removal of the solid components, are filtered aseptically and injected with suitable dilution into a subject for desensitizing purposes. The use of these aqueous extracts requires a substantial number of typically uncomfortable injections with gradually increasing amounts of allergen.

It is known that phenol in such injectable formulations can reduce the stability of the protein antigens, but subcutaneously injected glycerin-free products produce less stinging because they are not hypertonic. Although phenol-free formulations present fewer stability problems, phenol containing formulations remain the market leader for subcutaneous allergenic extracts. Market leaders produce injectable allergenic extracts typically with 0.4 percent phenol as the preservative.

The first exposure to an effective allergen typically causes only a mild immune response that sensitizes the immune system to the substance. Subsequent exposures to the allergen, however, can result in allergic symptoms, typically in a dose dependent manner (i.e., the allergen must reach a certain threshold), and may cause an increasingly severe response with repeated exposures. Allergic symptoms include, for example, itching and swelling of affected tissues, rashes, muscle spasms and other more severe symptoms. The type of symptom depends on the specific allergen, the part of the body where exposure occurs, and the degree of sensitization of the individual. Allergens that are inhaled often cause nasal congestion, itchy nose and throat, and mucus production. In highly allergic individuals or with higher doses of allergen, coughing, wheezing, or similar symptoms occur. In contrast, ingested allergens may tend to cause, for example, itching of the throat, vomiting, stomach cramps, diarrhea, and skin rashes or, in cases of strong sensitivity, shock. Eczema is also associated with allergies; a decrease in allergies often results in an improvement of eczema.

Allergy shots have proven useful in many cases to significantly and permanently relieve the extent of suffering experienced by allergic individuals. In fact, the current allergy shot approach is the only method that may be regarded as a curative means to reverse many types of allergic conditions. Early desensitization using the allergy shot approach to specific allergens has also proven somewhat effective against the occurrence of cross-reactive allergies to other substances. For example, a patient receiving allergy shots to treat allergenic rhinitis (e.g., hay fever) by desensitizing against pollen has a decreased risk of becoming allergic to cat hair or other common allergens.

Although allergy shots are currently the only means for treating the disease, rather than the symptoms, there are obvious disadvantages to this treatment as it is performed today. Conventional immunotherapy is lengthy, lasting from 2 to 5 years. When administered by conventional subcutaneous dosing under medical supervision, the therapy can be painful, expensive, inconvenient and not ideally suited for all patients. Conventional immunotherapy by subcutaneous injection may be ineffective in up to one-third of all allergy sufferers and may be discontinued prematurely in another one-third of allergic individuals. When dosed properly and continued for sufficient time, immunotherapy has long term effectiveness including absence of return of symptoms after discontinuation in the remaining third of the allergic population. Because no other known therapy includes the possibility of long term remission or cure of allergy, it remains of interest and potential significant clinical benefit to find methods that improve the long term compliance with therapy leading to a higher proportion of patients achieving the goal of permanent remission or desensitization of the allergic response.

The treatment duration for conventional immunotherapy is long and time consuming, usually comprising a total of 30 to more than 100 allergen injections. Because injected allergenic extract may cause severe systemic side effects including anaphylaxis , patients must remain in the doctor's office for at least 20 minutes an up to an hour after each injection for observation. Thus, medical and economic costs are very high for this type of treatment, in addition to patient discomfort.

Conventional allergy shot regimens have two dosing phases which are the build up phase and the maintenance phase. The duration of each of these phases should be tailor fitted to the individual, but typically the build up phase is about 20 to 24 injections. The first phase employs about 20 allergy shots. During this, phase the amount of allergen injected is increased with each dose, starting with minute amounts (as low as 0.01 μg). Injections of diluted extracts of the allergen are administered on a regular schedule, usually twice a week or weekly at first, in increasing doses until a maintenance dose of about 3 to 100 μg, or usually about 10 to 30 μg has been reached. This maintenance dose, reached after an interval up to 20 weeks, is then injected every 1 to 4 weeks for a period of 3 or more years.

It usually takes several months and may take up to 3 years to reach a maintenance dose. Patients may experience some relief within 6 months; however, if there is no benefit within 18 months, the shots are generally discontinued. After stopping immunotherapy, about one-third of allergy sufferers no longer have any symptoms, one-third have reduced symptoms, and one-third relapse completely.

In addition, during the desensitization phase, as more allergen is administered the injections usually cause moderate to sometimes severe side effects ranging from soreness and local swelling (wheal) or rash (flare) at the injection site to systemic allergic effects such as generalized skin rash or hives (urticaria), asthma, or even allergic shock (anaphylaxis). Other common side effects of immunotherapy include general itching (pruritis), red eyes and low blood pressure. Side effects usually occur within 20 minutes, although some can develop up to 2 hours after the allergy shot is given. Anaphylaxis refers to an allergic reaction characterized by a sharp drop in blood pressure, hives or welts, and breathing difficulties, that occurs immediately, progresses rapidly and is often life-threatening. Anaphylaxis is the most severe reaction that can result from standard immunotherapy or other exposure to such allergens.

As can be readily seen, subcutaneous dosing of allergenic extracts is characterized by relative inconvenience due to the requirement for patients to physically present to a professionally supervised location for regular injections. Moreover, the occurrence of adverse reactions is typical as the doses are increased.

Current methods of injection and sublingual allergen immunotherapy include giving increasing doses of an allergen to gradually build up a patient's tolerance to the allergen. Incremental doses are enabled by stepwise increases in both the concentration and volume of the allergen composition administered. In sublingual and oral immunotherapy, the allergen extract is typically given as drops, usually placed under the tongue and then swallowed. Convenience is a benefit of oral immunotherapy, because the patient can take the drops at home without the need for a physician or an office visit. Administration of allergenic extracts by sublingual routes has been described in the published literature as a means of reducing the inconvenience of the injected dosage regimen. Results of sublingual immunotherapy, however, have reported very high oral adverse event profiles. Esch, Robert E., et al., “Sublingual-oral administration of standardized allergenic extracts: phase 1 safety and dosing results,” Ann Allergy Asthma Immunol. 2008; 100: 475-481. Unpleasant taste is an adverse event which may reduce the degree of compliance and which could lead to rejection of appropriate therapy.

What is needed are methods and compositions for treatment of allergic reactions that are easier to administer than injection, with less pain and adverse side effect(s), and are effective for immunotherapy for the relief of allergies, yet achieve high patient compliance with the treatment regimen. Methods that do not require constant office visits or complete physician oversight would also be advantageous. Methods and compositions that are easily administered to a patient without the need for dilution to provide increasing amounts of allergen to the patient are also needed to increase use and acceptance by patients with allergy.

### SUMMARY OF THE INVENTION

The present invention relates to a method for treating a patient suffering from allergy by desensitizing the patient to an allergen component. The method includes orally, sublingually, or intranasally administering to the patient a therapeutically effective amount of an immunomodulating composition that includes an amount of the allergen component sufficient to impart an immune response and a pharmaceutically acceptable carrier that is at least substantially free of phenol, and in one embodiment is entirely free of phenol. In a preferred embodiment the pharmaceutically acceptable carrier is entirely free of phenol and includes a glycerin-based diluent. The method further includes successively repeating the administration at a selected interval. The therapeutically effective amount of each successive administration includes an increasing amount of the allergen component. The successive repeating occurs a sufficient number of times to reduce patient sensitivity to the allergen component, and the administering and repeat administering avoids or minimizes oral adverse effects.

In one embodiment, the glycerin-based diluent is present in an amount of about 35 to 65 percent (v/v) of the carrier. The pharmaceutically acceptable carrier may also include at least one of a buffer, water, sodium chloride or other electrolyte or a combination thereof. In a preferred embodiment, the diluent includes glycerin and at least one of sodium chloride or sodium bicarbonate. In one embodiment, the glycerin is present in an amount of about 25 to 75 percent (v/v), sodium chloride is present in an amount of about 0.1 to 10 percent (w/v), and sodium bicarbonate is present in an amount of about 0.025 to 2.5 percent (w/v), of the carrier. In an exemplary embodiment, the glycerin is present in an amount of about 50 percent (v/v), sodium chloride is present in an amount of about 1 percent (w/v), and sodium bicarbonate is present in an amount of about 0.25 percent (w/v), of the carrier.

In another embodiment, the pharmaceutically acceptable carrier further includes one or more parabens, such as methyl or ethyl paraben. The one or more parabens is typically present in an amount of about 0.1 to 1 percent (w/v) of the carrier. Antimicrobial preservation may also be achieved by minimizing or eliminating water from the final formulation to yield a composition that is substantially free of water.

The allergen component in the immunomodulating composition may be any suitable allergen, including, but not limited to, one or more pollens, weeds, epidermals, molds, dust, insects, venoms, or foods, or a combination thereof. In one embodiment, the epidermal includes one or more types of cat hair, cattle hair, horse hair, mouse hair, rabbit hair, guinea pig hair, hog hair, hamster hair, chicken feathers, duck feathers, or goose feathers, or a combination thereof. In another embodiment, the cat hair is present in a concentration of greater than about 10,000 BAU/mL, preferably about 10,000 to 100,000 BAU/mL, in the immunomodulating composition. The allergen component may advantageously be present in a concentration greater than that present in a standardized extract for the allergen.

The volume of the immunomodulating composition typically administered per day is about 0.01 mL to about 1.0 mL. Preferably, the daily volume is about 0.05 mL to 0.50 mL, and more preferably about 0.05 mL to 0.30 mL.

The immunomodulating composition may be administered in a dosage form known to those of ordinary skill in the art, including, but not limited to a solution, suspension, emulsion, tablet, lozenge, quick dissolving film or quick dissolving tablet, capsule, infused paper or fabric, nasal spray, or combination thereof. Further, the composition may be formulated for timed release.

The present invention also relates to a method for treating a patient suffering from allergy by desensitizing the patient to allergen component. The method includes providing a plurality of associated therapeutic doses for patient self-administration, and administering the doses orally, sublingually, or intranasally to the patient at progressively increasing concentrations of the allergen at subsequent time intervals. The doses of the allergen component are arranged from a lower concentration of the allergen to a higher concentration of the allergen to facilitate administration and patient compliance. The doses also include an immunomodulating composition that includes an amount of the allergen component sufficient to impart an immune response in the patient, and a pharmaceutically acceptable carrier including a glycerin-based diluent. The carrier is at least substantially free of phenol, and the administration has a minimal onset of oral adverse effects. In a preferred embodiment, the carrier is entirely free of phenol.

In one embodiment, the dose is in liquid drop or spray form, typically adapted for oral or nasal administration, and including but not limited to solutions, suspensions, and emulsions. In another embodiment, the doses are in the solid form of a tablet or capsule, lozenge, quick dissolving film, quick dissolving tablet, or a combination thereof. In another embodiment, the dose is in the form of a solid foundation such as an inherently inactive, non-therapeutic preformed tablet, film, fabric, paper or other framework upon which a liquid immunomodulating composition had been applied and dried so as to create an appropriately concentrated dose.

In one embodiment, the dose formed by coating a framework with immunomodulating composition may be mass produced in a single composition. In another embodiment, the framework with immunomodulating composition is manufactured using a patient-specific blend of antigens formulated at patient-specific optimal concentrations. In yet another embodiment, associated therapeutic doses include more than one identical dose. The associated doses, which may be provided in a single package, typically include at least two different doses of the allergen component. The doses are generally arranged for administration to have quantities of the allergen component that increase by a fixed increment compared to the preceding dose.

The present invention further relates to an immunomodulating product that includes a therapeutically effective amount of an immunomodulating composition and instructions for the treatment regimen. The composition includes an amount of the allergen component sufficient to impart an immune response in a patient and a pharmaceutically acceptable carrier that includes carrier that is at least substantially free of phenol. The instructions direct the patient to orally, sublingually, or intranasally administer the composition, and successively repeat the administration at a selected interval. The therapeutically effective amount of each successive administration preferably includes an increasing amount of the allergen component, and the successive repeating occurs a sufficient number of times to reduce patient sensitivity to the allergen component.

In one embodiment, the allergen component is present in a concentration greater than that present in an FDA standardized allergenic extract for the allergen. For example, the allergen component is made more concentrated than the FDA standardized allergenic extracts by formulating the extract at higher concentrations than currently approved for marketing. In another embodiment, the allergen component is made more concentrated that the FDA standardized allergenic extracts by evaporating or ultrafiltering some or all of the liquids from a standardized extract. In another embodiment, the evaporated or ultrafiltered extract is allowed to maintain a fluid characteristic, while in other embodiments, the evaporated or ultrafiltered extract is processed to a completely dried state which is then dosed by sublingual placement.

The immunomodulating composition may be packaged as single dose aliquots for antimicrobial preservation. In a preferred embodiment, the pharmaceutically acceptable carrier includes a glycerin-based diluent.

It should be understood that various embodiments described herein may be used in association with other embodiments described herein.

### DETAILED DESCRIPTION OF THE PREFERRED EMBODIMENTS

It has been surprisingly found that immunomodulating compositions adapted for non-injectable formulation that include an allergen component in a pharmaceutically acceptable carrier, at least substantially free of phenol, results in a better therapeutic profile preferably including less adverse effect compared to compositions that contain phenol. In particular, it has been discovered that the incidence of oral adverse effects can be reduced when compositions including at least substantially phenol-free carriers are administered orally or sublingually compared to injection of phenol-stabilized formulations. By “avoid or minimize oral adverse effects” or “minimal onset of oral adverse effects” is meant that the adverse effects are reduced compared to dosing of phenol-containing formulations.

Thus, the present invention includes methods and immunomodulating compositions for immunotherapy treatments leading to desensitization to allergens. The methods include administration of an allergen component to a patient that responds with an immune response, or responds in an allergic manner, to the allergen component. Preferably, the patient is human or an animal (e.g., a mammalian animal), more preferably a human. Routes of administration according to the invention include all of those known, including but not limited to, nasal, pulmonary, inhalation, mucosal, oral, sublingual, gastrointestinal, transdermal, electrophoresis, intra-rectal, and intra-vaginal, provided that the methods and compositions according to the invention are not injections. Preferred routes of administration are non-invasive and pain-free as compared to injections. In one embodiment, the composition is in liquid form and administered orally. In a preferred embodiment, the methods and compositions involve oral, sublingual, and/or intranasal administration. As used herein, “oral” or “orally” relates to administration of the composition through the mouth and swallowed, Typical oral dosage forms include pills, tablets, capsules, solutions, emulsions, and syrups. As used herein, “sublingual” or “sublingually” relates to administration of the composition underneath the tongue. A typical sublingual dosage form is a solid dosage form such as a lozenge put under the tongue where it dissolves and is presented to local tissue, but could also include a spray or other dosage form. As used herein, “intranasal” or “intranasally” relates to administration within the nose. A typical intranasal dosage form is an aerosol spray or liquid drop.

Immunotherapy and the methods according to the present invention are thus effective in the treatment or management of allergies, or one or more symptoms thereof, including, but not limited to, allergic asthma, allergic rhinitis, and stinging insect hypersensitivity. Food allergies can also be treated with the present invention. Allergen immunotherapy may prevent the development of asthma in children with allergic rhinitis.

Systemic allergic reactions common after administration of an allergen component include, but are not limited to, allergic rhinitis or conjunctivitis (swelling and redness of nasal or eye membranes), respiratory problems (difficulty breathing), abdominal cramps, severe gastrointestinal upset (vomiting), weakness, shock/unconsciousness, hypotension or fainting, and difficulty breathing/laryngeal blockage (massive swelling in the throat). Surprisingly, administration of compositions at least substantially free of phenol according to the invention does not generate a significant adverse effect.

“Adverse effect” refers to one or more oral adverse events or other adverse effects of administering the immunomodulating compositions administered according to the invention. By “oral adverse effects” is meant allergy-like adverse events affecting the mouth, nose, and throat, including, but not limited to, itchy throat, itchy nose, runny nose, stuffy nose, itchy mouth, coughing, hay fever, severe sneezing, sore throat, vomiting and bad taste in mouth. The term “oral adverse effects” encompasses allergy-like oropharyngeal and nasal adverse events, and bad taste in mouth which could lead to non-compliance or rejection of appropriate therapy.

The present invention provides immunomodulating compositions that include a pharmaceutically acceptable carrier that at least substantially free of phenol. By “substantially free” is meant less than about 0.2 percent (w/v) phenol, preferably less than about 0.1 percent (w/v), and more preferably less than about 0.05 percent (w/v) phenol; based on the pharmaceutically acceptable carrier. In an exemplary embodiment, the composition is entirely free of phenol. Similarly, by “substantially free of water” is meant less than about 0.2 percent water, preferably less than about 0.1 percent water, and more preferably less than about 0.05 percent water, based on the pharmaceutically acceptable carrier. In one embodiment, the immunomodulating composition that is administered is entirely free of water.

In a preferred embodiment, the immunomodulating compositions are glycerin based. By “glycerin-based” is meant that glycerin is present in an amount of at least about 20 percent (v/v), preferably at least 40 percent (v/v), and more preferably at least about 50 percent (v/v), of the pharmaceutically acceptable carrier. Glycerin is typically present in an amount of about 35 to 65 percent (v/v) of the pharmaceutically acceptable carrier. In alternative embodiments neither phenol nor glycerin are used and antimicrobial preservation is afforded by one or more of alternative preservatives, such as the parabens (e.g., methyl- and ethylparaben) or salts (e.g., sodium benzoate). In one embodiment, the carrier is substantially free of all antimicrobial preservatives, this made possible by dessication of the final dosage form or production of single dose dosage forms. Thus, in all embodiments the carrier is at least substantially free, preferably entirely free of phenol, and may be stabilized by the incorporation of glycerin or parabens, or through the use of dried dosage forms, or through presentation as single dose dosage forms, or any combination thereof.

Without being bound by theory, glycerin acts as one or more of a solvent, humectant, emollient, plasticizer, and sweetener. When added to immunomodulating compositions, it may act as a protein stabilizer, protease inhibitor, and bacteriostatic. The compositions used in the present invention are advantageously both stabilized and rendered bacteriostatic through the use of glycerin while remaining at least substantially free of phenol.

Without being bound by theory, it is believed that the absence of phenol from the compositions may advantageously and surprisingly provide the basis for the minimal adverse effect shown according to the invention. While the phenol-based formulations demonstrated fewer adverse effects upon subcutaneous dosing, the glycerin-based formulations of the invention demonstrated superiority of therapeutic profile on oral, sublingual, and intranasal dosing. Glycerin containing formulations are reportedly painful when injected. This hypertonicity does not, however, represent a problem for mouth contact surfaces as presently discovered, unlike phenol conventionally used in oral and sublingual immunomodulating compositions. The term “therapeutic profile” is meant to encompass various factors, including dosage amount and frequency, efficacy, adverse effect, etc.

Phenol in concentrations more than 5 times higher than used in typical allergenic extracts is present in Chloraseptic® sore throat spray. Although not believed to be marketed via NDA or possessing an OTC monograph, this sore throat spray remains on the U.S. market. It is believed that phenol in such product induces a mouth feel that, while apparently anesthetic in the face of sore throat, might be perceived similarly to potential allergic adverse reactions when administered in support of oral, sublingual, or intranasal immunotherapy.

This unpredicted difference in relative behavior under oral, sublingual, or intranasal dosing compared to subcutaneous dosing would have utility in improving the therapeutic profile, or at least in reducing adverse effect including undesirable local tissue sensations, after oral, sublingual, or intranasal therapy. This difference is important in and of itself, but could have additional clinical importance if the adverse effects associated with phenol inclusion in an oral, lingual, or intranasal formulation are difficult to distinguish from those which might signal a problematic reaction to the allergen.

The glycerin-based pharmaceutically acceptable carrier herein may include one or more other excipients and adjuvants that aid in administration, In a preferred embodiment, the carrier will include at least one of water, a buffer, saline, or a combination thereof. In a preferred embodiment, the carrier includes glycerin and at least one other component, such as sodium chloride and sodium bicarbonate, or a combination thereof. In one embodiment, glycerin is present in an amount of about 20 to 80 percent (v/v), preferably 25 to 75 percent (v/v), and more preferably 50 to 55 percent (v/v) of the pharmaceutically acceptable carrier. Sodium chloride is generally present in an amount of about 0.01 to 15 percent (w/v), preferably about 0.1 to 10 percent (w/v), and more preferably about 0.5 to 0.9 percent (w/v) of the carrier. The preferred buffer is sodium bicarbonate, and it is typically present in an amount of about 0.01 to 10 percent (w/v), preferably about 0.025 to 2.5 percent (w/v), and more preferably about 0.1 to 0.5 percent (w/v) of the carrier. In an exemplary embodiment, the glycerin is present in an amount of about 50 percent (v/v), sodium chloride is present in an amount of about 1 percent (w/v), and sodium bicarbonate is present in an amount of about 0.25 percent(w/v), of the carrier. For example, the carrier can include 52.5 percent (v/v) glycerin, 0.95 percent (w/v) sodium chloride, and 0.24 percent (w/v) sodium bicarbonate, with the rest being water. In one embodiment, glycerin is present in a hypertonic concentration such as those described above but is diluted at the time of dosing to create an isotonic, or slightly hypertonic solution for dosing by intranasal routes.

The immunomodulating compositions include an allergen component, that includes one or more allergens that cause an allergic reaction in a patient. An allergen may be in the form of an allergenic extract, purified native or recombinant allergen, a modified allergen, or a nucleic acid that encodes the allergen. As used herein, “allergen” refers to any material to which a patient with a functioning immune system can mount an immune response, such as that mediated by T cells, B cells, mast cells and any other cells or combination thereof, and the term “allergen” can be used interchangeably with the terms immunogen, antigen, epitope, and includes fragments and whole particles.

The phrase “therapeutically” in connection with the effective amount includes that amount of allergen component that provides an immune response and a therapeutic benefit in the treatment or management of an allergic reaction, or one or more symptoms or conditions associated therewith. In one embodiment, the allergenic component of one or more allergens is administered in an amount that is at least about one times to 500 times, preferably at least about 10 times, and more preferably at least about 30 times the injection amounts for the one or more allergens. The compositions herein can be self-administered through methods of oral, sublingual, or intranasal routes, are beneficial to patients, and also allow for ease of administration of an allergenic component including multiple allergens: This is relevant because the induction and maintenance of immunological tolerance requires relatively high allergen doses that can be difficult to attain when multiple allergens are included in the immunotherapy formulation.

The dose of allergen component administered typically ranges from nanograms to milligrams of allergen provided to the patient, depending on the allergen, the route of administration and the reactions of the individual patient's immune system. The dose is readily determined by one of ordinary skill in the art, particularly based on the guidance herein. In general, for a conventional injection administration route, the starting injection immunotherapy dose is typically 10 to 100-fold less, most often 25 to 50-fold less than the maintenance dose. While injected therapy is seldom advanced more frequently than one gradation per week, oral, sublingual or nasal dosing can be undertaken with more frequent advancement of dose, with incremental dose advancement possible on a daily, or multiple dose per day regimen. The use of an oral, sublingual, or nasal route of administration according to the invention allows for significantly higher cumulative doses over a shorter time period because of more frequent dosing that is allowed by self-administration and the safety profile afforded by the oral or sublingual route of administration.

Conventional maintenance injection therapy for short ragweed pollen or cat hair allergen is given at less frequent intervals, typically increasing over a period time to once monthly injections of the highest concentration allergen solution. The maximum doses that can be administered during such conventional injection immunotherapy are often limited by safety considerations, and by time available to visit a physician's office to receive the injections. Based on the recommended dose schedule described above and taken from a conventional product package insert, the monthly cumulative maintenance dose of allergen is about 15 μg. In contrast, sublingual, oral, or nasal maintenance immunotherapy allows for monthly cumulative maintenance doses of 30 or more times that given by traditional injection immunotherapy.

In one embodiment, the concentration of immunomodulating allergen is increased over subsequent doses. This can be brought about through the provision of more than one multi-dose containers each with different concentrations of allergen per unit volume or through multiple single-dose containers with a sequential increment in allergen concentration.

An exemplary embodiment of the method of the present invention includes administering an allergen component in increasing dosage amounts, which increases the amount of allergen(s) administered, from a source having a uniform concentration. The concentrations of the one or more allergens in the allergen component are not altered due to treatment regimen requirements for diluted allergen amounts for the initial doses for administration to patients. The methods advantageously thus allow for the oral, sublingual, or intranasal administration of a single concentration of an allergen component. Doses of a single, uniform specific concentration may be administered, without further dilution. For example, in an oral dosing administration of a liquid composition, the dose can include one or more drops of a liquid having a specific concentration of the allergen component. For other dosage formulations, for example, quick dissolving tablets, a dose could be a quick dissolving tablet, and all of the tablets provided would have a uniform or specific concentration of the allergen component. In one embodiment of an immunomodulating composition, the concentration of allergen component does not typically increase, but remains uniform or constant. Instead, the volume or the mass of the dosage faun, e.g., a tablet, could increase or decrease to obtain the desired therapeutic amount at that time or after a given time interval, or a user could take multiple tablets to obtain the desired concentration.

In one embodiment, in the initial dosing interval, the patient can be provided with a container of a liquid immunomodulating composition having a particular concentration of allergen component. The patient can administer the anti-allergenic composition via one of the oral, sublingual, or intranasal routes in a prescribed volume that can be modified in an increasing step-wise fashion over the initial dosing period. In one embodiment, a plurality of graduated, measured containers each including this step-wise increased or decreased dose can be provided into which the primary container can dispense the desired dose. For the second, subsequent dosing interval, the patient may be provided with a second container of an immunomodulating composition. The second container includes an immunomodulating composition wherein the concentration of allergen component is preferably the same as the first container used in the initial dosing interval. Again, the patient administers the anti-allergenic composition via one of the oral, sublingual, or intranasal routes in prescribed volume that is increased or decreased in a step-wise fashion over the second dosing interval. There may be one or multiple dosing intervals in a treatment regimen. The step-wise change in the amount of one or more allergens administered during a dosing interval is accomplished by step-wise increases or decreases in the delivered volume of the anti-allergenic composition of that interval. In one preferred embodiment the step-wise modification is an increased dose.

Thus, by way of example as to the dosing containers, a first example is to provide a plurality of differently-sized containers associated to form the needed dosage amounts during a treatment regimen, or portion thereof, with each container having the same concentration of anti-allergenic concentration therein. In a second example, a single container with all the needed dosage in a single concentration could be provided, and the varied dose amount can be dispensed such as with one or more pushes of a pump in the case of a liquid or one or more tablets in the case of capsules or tablets of uniform concentration and size. In this second example, a patient can or might take one solid dosage form on Day 1, two on Day 2, etc. In an alternative embodiment, a set of graduated, measured containers can be included along with the container of uniformly concentrated dose, to facilitate measurement during the treatment regimen. In yet another alternative embodiment, the dispensing mechanism can be adapted to provide an increased amount with each successive dispensing, either automatically or manually by the user. In each of these two examples, the use of containers of different concentrations can be used as discussed above; however, preferably as few containers of different concentrations as possible are prepared and administered.

The use of a single concentration of an immunomodulating composition to accomplish dose adjustments during allergen immunotherapy can afford an additional significant manufacturing advantage over the use of two or more different concentrations, as is typically currently practiced. In an alternative embodiment, however, about 2 to 5 containers, preferably about 2 to 3 containers, each with different concentrations can be used to minimize the need for a different concentration of each dose in a complete treatment regimen, thereby gaining a portion of the advantage of using a single concentration composition. The combination of methods involving different concentrations of allergen per each container and delivery of different volumes immunomodulating composition with a constant concentration affords the ability to span large multiples of difference between initial dose and maintenance dose.

The methods of the present invention can include treatment regimens that include single or multiple dosing intervals, each of which provides step-wise changes in the dose of allergen component that are administered over a particular time period and at successive time intervals, preferably of equal size. In the case of a single dosing interval, this refers to an initial dose administered on Day 1 and a final dose administered after a specific period of time, so that only two doses are administered. The intervals can range from hours to days to weeks to months. For example, the initial dose can be administered on Day 1, and the next dose administered on Day 2, Day 8, or Day 16. The dosing intervals can be anywhere between a few hours to a few days to one week to a few months over a course of a year. Typically, the dosing interval is no more than about one week, preferably no more than about 5 days, and more preferably no more than about one day. In exemplary embodiments, the dosing interval is no more than about 12 hours, or no more than about 8 hours. In one embodiment, the selected intervals all occur within about six months to five years, preferably about one to three years.

The dosing intervals may be altered depending on the allergen(s) in the allergen component, the route of administration, and the reactions of the patient's immune system. For example, the methods of the present invention may include rush or ultra-rush immunotherapy, wherein a more rapid, or rushed, dosing interval is used to reach the maintenance dose of the allergen component. During the initial phase of treatment, increasing doses of allergen may be given about 30 minutes to about 2 to 4 hours apart, rather than every few days or weeks. Patients may be pre-treated with medications to reduce the risk of an allergic reaction during rush immunotherapy. In an ultra-rush regimen, the maintenance dose can be safely reached in about 2 to 3 days or less without increased systemic adverse side effect. In addition, oral anti-allergy medications such as oral dosage forms of antihistamines, topical corticosteroids, leukotriene inhibitors, or mast cell stabilizers can be administered in association with the anti-allergen compositions (i.e., concurrently or sequentially), or even in the same formulation with the allergen component and carrier.

An example of a rush immunization schedule could start with Day 1 having 2 drops twice a day (e.g.,100 AU/day), building up on Day 2 to 4 drops twice a day and on Day 3 to 6 drops twice a day with the drops each at the same concentration, followed by a maintenance amount of about 8 to 10 drops twice a day (e.g., 400-500 AU/day) for a continuous period.

The methods of the present invention include administering a predetermined minimum dose, as delivered by a specified amount of the anti-allergenic composition, for a specified time period, such as one or more days, and increasing the dose of the immunomodulating composition by increasing the amount of the composition given to a patient until the maximum dose is reached so that immune tolerance is more readily induced and maintained. Induction of immune tolerance includes the initial administration and any increasing dose amounts until a maximum amount is administered and the patient does not have adverse effects. The maximum amount can be the maintenance dose. For example, oral doses are provided in increasing amounts of allergen until the patient indicates discomfort in the oral cavity or has adverse immune responses to that level of allergen. At that point, the amount of allergen delivered is decreased until no such symptoms are reported or measured. Maintenance immune tolerance levels are attained by providing allergen at a level where the patient does not report adverse or uncomfortable symptoms, and this level can be measured by analyzing for a decrease in allergen-specific IgE antibodies or reaction to allergen challenge and an increase in allergen-specific IgG or IgA antibodies or Th2-type cytokine secretion by allergen-specific T cells.

In one embodiment, a dose of the immunomodulating composition can be administered to a patient (e.g., human or animal), and the immune tolerance to the allergen component is induced and maintained. The allergic response to the allergen component is generally measured by skin testing. Inducing and maintaining immune tolerance is generally determined by a reduction in response to the allergen by the patient. Such tests are known to those of ordinary skill in the art. The administration of the composition includes providing a predetermined dose (e.g., a minimum number of drops) on the first day of treatment and increasing the dose in the succeeding days until a maximum dose is administered per day. Alternatively, administering may include providing a predetermined minimum dose (e.g., a number of drops) on the first day of treatment and providing the maximum dose on each day thereafter. Depending on patient needs, the maximum dose may be reduced when environmental exposure to allergen component is increased.

The immunomodulating compositions of the present invention include an allergen component including one or more allergens, including but not limited to, cat hair, house dust mite, dusts, molds, weeds, grass pollen, short ragweed pollen, mixtures or combinations thereof. Particular compositions include a standardized cat hair extract (e.g., 10,000 AU/mL), a standardized house dust mite extract (e.g., 10,000 AU/mL), a standardized grass pollen extract (e.g., 100,000 BAU/mL), and a standardized short ragweed pollen extract (e.g., 1:20 w/v). Other combinations may depend upon the allergic profile of allergic patients determined by a qualified medical practitioner and the identification of one or more specific triggers of the patient's reaction or symptoms. Inclusion criteria may be based on allergen skin testing or allergen-specific IgE measurements, or both.

The compositions of the present invention can advantageously include a concentration of allergen greater than that in standardized extracts. For example, immunomodulating compositions with the allergen component including cat hair can be formulated to have a concentration greater than about 10,000 BAU/mL of cat hair. Greater concentrations of allergen component reduce the volume or mass of the dosage form administered, making the appropriate dose easier to administer. Doses can be driven to an even higher concentration without increasing the volume of drug administered. The cat hair extract may typically include about 10,000 BAU/mL to 100,000 BAU/mL, or in varying embodiments from about 12,000 BAU/mL to 80,000 BAU/mL, from about 15,000 BAU/mL to 75,000 BAU/mL, from about 20,000 BAU/mL to 50,000 BAU/mL of cat hair, or any other appropriate amount within these ranges or combinations thereof. Similar amounts may be used for other allergens described herein, or adjusted as appropriate by one of ordinary skill in the art. For example, the volume of the immunomodulating composition that is administered per day is typically about 0.01 mL to about 1 mL, preferably about 0.05 mL to 0.50 mL, more preferably 0.05 mL to 0.30 mL when in liquid form. A dropper or volumetric pump can be made to deliver about 0.05 mL so this would be about 1 to 10 drops per day, preferably 1 to 6 drops per day. With a higher concentration or with a larger delivered drop size, the number of drops administered would be fewer.

The immunomodulating compositions of the present invention include an allergen component, and a pharmaceutically acceptable carrier including the glycerin-based pharmaceutical carrier and optionally one or more additional pharmaceutical agents or excipients typically used to provide a desired dosage form and that are suitable given the selected allergen(s) in the allergen component. The allergen component preferably includes one or more of the following allergens or a derivative thereof that include, but are not limited to: pollens (e.g., farm plant, tree, weed, grass), animal danders, fungi, hymenoptera venoms, insects and house dust mites, plant foods, and animal foods.

The allergen can be selected from one or more types of mites, e.g., Mite, House Dust (Dermatophagoides farinae); Mite, House Dust (Dermatophagoides pteronyssinus); Mite, Food/Storage (Acarus siro); Mite, House Dust (Blomia tropicalis); Mite, Storage (Chortoglyphus arcuates); Mite, House Dust (Euroglyphus maynei); Mite, Food/Storage (Lepidoglyphus destructor); Mite, Food/Storage (Tyrophagus putrescentiae); and Mite, House Dust (Glycyphagus domesticus).

The allergen can be selected from one or more types of venoms, e.g., Bumble Bee Venom (Bombus spp.); European Hornet Venom (Vespa crabro); Honey Bee (Apis mellifera.); Mixed Hornet Venom (Dolichovespula spp); Mixed Paper Wasp Venom (Polistes spp.); Mixed Yellow Jacket Venom (Vespula spp.); White (bald)-faced Hornet Venom (Dolichovespula maculate); and Yellow Hornet Venom (Dolichovespula arenaria).

The allergen can be selected from one or more types of insects, e.g., Ant, Carpenter (Camponotus pennsylvanicus); Ant, Fire (Solenopsis invicta); Ant, Fire (Solenopsis richteri); Cockroach, American (Periplaneta Americana); Cockroach, German (Blattella germanica); Cockroach, Oriental (Blatta orientalis); Horse Fly (Tabanus spp.); House Fly (Musca domestica); Mayfly (Ephemeroptera spp.); Mosquito (Culicidae sp.); and Moth (Heterocera spp.).

The allergen can be selected from one or more types of epithelia, dander, and hair and feathers, e.g., Canary Feathers (Serinus canaria); Cat Epithelia (Felis domesticus)); Cattle Epithelia (Bos Taurus); Chicken Feathers (Gallus gallas (domesticus)); Dog Epithelia, Mixed Breeds (Canis familiaris); Duck Feathers (Anal platyrhynchos); Gerbil Epithelia (Meriones unguiculatus); Goat Epithelia (Capra hircus); Goose Feathers (Anser domesticus); Guinea Pig (Cavia porcellus); Epithelia ((cobaya)); Hamster Epithelia (Mesocricetus auratus); Hog Epithelia (Sus scrofa); Horse Epithelia (Equus caballus); Mouse Epithelia (Mus musculus); Parakeet Feathers (Psittacidae spp.); Pigeon Feathers (Columba fasciata); Rabbit Epithelia (Oryctolagus cuniculus); Rat Spithelia (Rettus norvegicus); and Wool, Sheep (Ovis aries).

The allergen can be selected from one or more types of dander, e.g., Cat dander/Antigen (Felis catus (domesticus)); Dog Dander, Mixed-Breed (Canis familiaris); and Poodle Dander (Canis familiaris).

The allergen can be selected from one or more types of fungi, e.g., Acremonium strictum; Alternaria alternate; Aspergillus amstelodami; Aspergillus flavus; Aspergillus furmigatus; Aspergillus nidulans; Aspergillus niger; Aspergillus terreus; Aspergillus versicolor; Aureohasidium Pullulans; Bipolaris sorokiniana; Botrytis cinerea; Candida albicans; Chaetomium globosum; Cladosporium herbarum; Cladosporium sphaerospermum; Drechslere spicifera; Epicoccum nigrum; Epidermophyton floccosum; Fusarium moniliforme; Fusarium solani; Geotrichum candidum; Gliocladium viride; Helminthosporium solani; Microsporum canis; Cephalosporium acremonium; Alternaria fermis; Aspergillus glaucus; Pullularia pullulans; Drechslera sorokiniana; Helminthosporium sativum; Hormodendrum hordei; Curvularia spicifera; Epicoccum purpurascens; Oospora lactic; Gliocladium deliquescens; Spondylocladium atrovirens; Microsporum lanosum; Mucor circinelloides f. circinelloides; Mucor circinelloides f. lusitanicus; Muncor plumbeus; Mycogone perniciosa; Neurospora intermedia; Nigrospora oryzae; Paecilomyces variotii; Penicillium brevi-compactum; Penicillium camembertii; Penicillium chrysogenum; Penicillium digitatum; Penicillium expensum; Penicillium notatum; Penicillium roquefortii; Phoma betae; Phomma herbarum; Rhigopus oryzae; Rhizopus stolonifer; Rhodotorula mucilaginosa; Saccharomyces cerevisiae; Scopulariopsis brevicaulis; Serpula lacrymans; Setosphaeria rostrata; Stemphylium hotryosum; Stemphylium solani; Trichoderma harzianum; Trichophyton mentagrophytes; Trichophyton rubrum; Trichothecium roseum; Mucor mucedo; Mucor racemosus; Neurospora sitophil; Monilia sitophila; Phoma pigmentivora; Rhizopus arrhizus; Rhizopus nigricans; Rhodotorula rubra var. mucilaginosa; Merulius lacrymans; Exserohilum rostratum; Helminthosporium halodes; Trichoderma viride; Trichophyton interdigitale; and Cephalothecium roseum.

The allergen can be selected from one or more types of smuts, e.g., Barley Smut (Ustilago nuda); Bermuda Grass (ustilago); Smut (cynodontis); Corn Smut (Ustilago maydis); Johnson Grass (Sporisorium); Smut (cruentum); Oat Smut (Ustilago avenae); and Wheat Smut (Ustilago tritici).

The allergen can be selected from one or more types of grass pollens, e.g., Bahia (Paspalum notalum); Bermuda (Cynodon dactylon) Blue, Canada (Poa compressa); Brame, Smooth (Bromus inermis); Canary (Phalaris arundinacea); Corn (Zea mays); Couch/Quack (Elytrigia repens (Agropyron repens)); Johnson (Sorghum halepense); Kentucky Blue (Poa pratensis); Meadow Fescue (Festuca pratensis (elatior)); Oat, Cultivated (Avena sativa); Orchard (Dactylis glomerata); Red Top (Agrostis gigantean (alba)); Rye, Cultivated (Secale cereale); Rye, Giant Wild (Leymus (Elymus) condensatus); Rye, Italian (Lolium perenne ssp. Multiflorum); Rye, Perennial (Lolium perenne); Sweet Vernal (Anthoxanehum odoratum); Timothy (Phleum pretense); Velvet (Holcus lanatus); Wheat, Cultivated (Triticum aestivum); St. Augustine grass (Stenotaphrum secundatum), and Wheatgrass, Western (Elymus (Agropyron).

The allergen can be selected from one or more types of weed pollens, e.g., Allscale (Atriplex polycarpa); Baccharis (Baccharis halimifolia); Baccharis (Baccharis sarothroides); Burrobrush (Hymenoclea salsola); Careless Weed (Amaranthus hybridus); Cocklebur (Xanthium strumarium (commune)); Dock, Yellow (Rumex crispus); Dog Fennel (Eupatorium capillifolium); Goldenrod (Solidago spp.); Hemp, Western Water (Amaranthus tuberculatus (Acnida tamariscina)); Iodine Bush (Allenrolfea occidentalis); Jerusalem Oak (Chenopodium botrys); Kochia/Firebush (Kochia scoparia); Lambs Quarter (Chenopodium album); Marsh Elder, Burweed (Iva xanthifolia); Marsh Elder, Narrowleaf (Iva angustifolia); Marsh Elder, Rough (Iva annua (ciliata)); Mexican Tea (Chenopodium ambrosioides); Mugwort, Common (Artemisia vulgaris); Mugwort, Darkleaved (Artemisia ludoviciana); Nettle (Urtica dioica); Palmer's Amaranth (Amaranthus palmeri); Pigweed, Redroot/Rough (Amaranthus retroflexus); Pigweed, Spiny (Amaranthus spinosus); Plantain, English (Plantago lanceolata); Poverty Weed (Iva axillaris); Quailbrush (Atriplex lentiformis); Rabbit Bush (Ambrosia deltoidea); Ragweed, Desert (Ambrosia dumosa); Ragweed, False (Ambrosia acanthicarpa); Ragweed, Giant (Ambrosia trifida); Ragweed, Short (Ambrosia artemisiifolia); Ragweed, Slender (Ambrosia confertiflora); Ragweed, Southern (Ambrosia bidentata); Ragweed, Western (Ambrosia psilostachya); Russian Thistle (Salsola kali (pestifer)); Sage, Coastal (Artemisia californica); Sage, Pasture (Artemisia frigida); Sagebrush, Common (Artemisia tridentate); Saltbush, Annual (Atriplex wrightii); Shadscale (Atriplex confertifolia); Sorrel, Red/Sheep (Rumex acetosella); and Wingscale (Atriplex canescens); Wormwood, Annual (Artemisia annua).

The allergen can be selected from one or more types of tree pollens, e.g., Acacia (Acacia spp.); Alder, European (Alnus glutinosa); Alder, Red (Alnus rubra); Alder, Tag (Alnus incana ssp. Rugosa): Alder, White (Alnus rhombifolia); Ash, Arizona (Fraxinus velutina); Ash, Green/Red (Fraxinus pennsylvanica); Ash, Oregon (Fraxinus latifolia); Ash, White (Fraxinus Americana); Aspen (Populus tremuloides); Bayberry (Myrica cerifera); Beech, American (Fagus grandifolia (americana)) Beefwood/Australian Pine (Casuarina equisetifolia); Birch, Black/Sweet (Betula lenta); Birch, European White (Betula pendula); Birch, Red/River (Betula nigra); Birch, Spring (Betula occidentalis (fontinalis)); Birch, White (Betula populifolia); Box Elder (Acer negundo); Cedar, Japanese (Cryptomeria japonica); Cedar, Mountain (Juniperus ashei (sabinoides)); Cedar, Red (Juniperus virginiana); Cedar, Salt (Tamarix gallica); Cottonwood, Black (Populus balsamifera ssp. Trichocarpa); Cottonwood, Eastern (Populus deltoids); Cottonwood, Fremont (Populus fremontii); Cottonwood, Rio Grande (Populus wislizeni); Cottonwood, Western (Populus monilifera (sargentii)); Cypress, Arizona (Cupressus arizonica); Cypress, Bald (Taxodium distichum); Cypress, Italian (Cupressus sempervirens); Elm, American (Ulmus Americana); Elm, Cedar (Ulmus crassifolia); Elm, Siberian (Ulmus pumila); Eucalyptus (Eucalyptus globulus); Hackberry (Celtis occidentalis); Hazelnut (Corylus Americana); Hazelnut, European (Corylus avellana); Hickory, Pignut (Carya glabra); Hickory, Shagbark (Carya ovata); Hickory, Shellbark (Carya laciniosa); Hickory, White (Carya alba); Juniper, Oneseed (Juniperus monosperma); Juniper, Pinchot (Juniperus pinchotii); Juniper, Rocky Mountain (Juniperus scopulorum); Juniper, Utah (Juniperus osteosperma); Juniper, Western (Juniperus occidentalis); Locust Blossom, (Robinia); Black (pseudoacacia); Mango Blossom (Mangifera indica); Maple, Coast (Acer macrophyllum); Maple, Red (Acer rubrum); Maple, Silver (Acer saccharinum); Maple, Sugar (Acer saccharum); Melaleuca (Melaleuca quinquenervia (leucadendron)); Mesquite (Prosopis glandulosa (julifiora)); Mulberry, Paper (Broussonetia papyrifera); Mulberry, Red (Morus rubra); Mulberry, White (Morus alba); Oak, Arizona/Gambel (Quercus gambeiji); Oak, Black (Quercus velutina); Oak, Bur (Quercus macrocarpa); Oak, California Black (Quercus kelloggii); Oak, California Live (Quercus agrifolia); Oak, California White/Valley (Quercus lobata); Oak, English (Quercus robur); Oak, Holly (Quercus ilex); Oak, Post (Quercus stellata); Oak, Red (Quercus rubra); Oak, Scrub (Quercus dumosa); Oak, Virginia Live (Quercus virginiana); Oak, Water (Quercus nigra); Oak, Western White/Gany (Quercus garryana); Oak, White (Quercus alba); Olive (Olea europaea); Olive, Russian (Elaeagnus angustifolia); Orange Pollen (Citrus sinensis); Palm, Queen (Arecastrum romanzoffianum (Cocos plumosa)); Pecan (Carya illinoensis); Pepper Tree (Schinus molle); Pepper Tree/Florida Holly (Schinus terebinthifolius); Pine, Loblolly (Pinus taeda); Pine, Eastern White (Pinus strobus); Pine, Longleaf (Pinus palustris); Pine, Ponderosa (Pinus ponderosa); Pine, Slash (Pinus elliottii); Pine, Virginia (Pinus virginiana); Pine, Western White (Pinus monticola); Pine, Yellow (Pinus echinata); Poplar, Lombardy (Populus nigra); Poplar, White (Populus alba) Privet (Ligustrum vulgare); Sweet Gum (Liquidambar styraciflua); Sycamore, Eastern (Platanus occidentalis); Sycamore, Oriental (Platanus orientalis); Sycamore, Western (Platanus racemosa); Sycamore/London Plane (Platanus acerifolia); Walnut, Black (Juglans nigra); Walnut, California Black (Juglans californica); Walnut, English (Juglans regia); Willow, Arroyo (Salix lasiolepis); Willow, Black (Salix nigra); and Willow, Pussy (Salix discolor).

The allergen can be selected from one or more types of wild and cultivated flowers, e.g., Daisy, Ox-Eye (Chrysanthemum leucanthemum); Dandelion (Taraxacum officinale); and Sunflower (Helianthus annuus).

The allergen can be selected from one or more types of cultivated farm plant pollens, e.g., Alfalfa (Medicago sativa); Castor Bean (Ricinus communis); Clover, Red (Trifolium pratense); Mustard (Brassica spp.); and Sugar Beet (Beta vulgaris).

The allergen can be selected from one or more types of plant food, e.g., Almond (Prunus dulcis); Apple (Malus pumila); Apricot (Prunus armeniaca); Banana (Musa paradisiaca (sapientum)); Barley (Hordeum vulgare); Bean, Lima (Phaseolus lunates); Bean, Navy (Phaseolus vulgaris); Bean, Pinto (Phaseolus sp.) Bean, Red Kidney (Phaseolus sp.); Bean, String/Green (Phaseolus vulgaris); Blackberry (Rubus allegheniensis); Blueberry (Vaccinium sp.); Broccoli (Brassica oleracea var. botrytis); Buckwheat (Fagopyrum esculentum); Cabbage (Brassica oleracea var. capitata); Cacao Bean (Theobroma cacao); Cantaloupe (Cucumis melo); Carrot (Daucus carota); Cauliflower (Brassica oleracea var. botrytis); Celery (Apium graveolens var. dulce); Cherry (Prunus sp.); Cinnamon (Cinnamomum verum); Coffee (Coffee Arabica); Corn (Zea mays); Cranberry (Vaccinium macrocarpon); Cucumber (Cucumis sativus); Garlic (Allium sativum); Ginger (Zingiber officinale); Grape (Vitis sp.); Grapefruit (Citrus paradise); Hops (Humulus lupulus); Lemon (Citrus limon); Lettuce Malt (Lactuca sativa); Mushroom (Agaricus campestris); Mustard (Brassica sp.); Nutmeg (Myristic fragrans); Oat (Avena sativa); Olive, Green (Olea europaea); Onion (Allium cepa var. cepa); Orange (Citrus sinensis); Pea, Blackeye (Vigna unguiculata); Pea, Green (Pisum sativum (English)); Peach (Prunus persica); Pear (Pyrus communis); Pepper, Black (Piper nigrum); Pepper, Green (Capsicum annuum var. annuum); Pineapple (Ananas comosus); Potato, Sweet (Ipomoea batatas); Potato, White (Solanum tuberosum); Raspberry (Rubus idaeus var. idaeus); Rice (Oryza sativa); Rye (Secale cereale); Sesame Seed (Sesamum orientale (indicum)); Soybean (Glycine max); Spinach (Spinacia oleracea); Squash, Yellow (Cucurbita pepo var. melopepo); Strawberry (Fragaria chiloensis); Tomato (Lycopersicon esculentum (lycopersicum)); Turnip (Brassica rapa var. rapa); Vanilla Bean (Vanilla planifolia); Watermelon (Citrullus lanatus var. lanatus); and Wheat, Whole (Triticum aestivum).

The allergen can be selected from one or more types of fish and shellfish, e.g., Bass, Black (Micropterus sp.); Catfish (Ictalurus punctatus); Clam (Mercenaria mercenaria); Codfish (Gadus morhua); Crab (Callinectes sapidus); Flounder (Platichthys sp.); Halibut (Hippoglossus sp.); Lobster (Homarus americanus); Mackerel (Scomber scombrus); Oyster (Crassostrea virginica); Perch (Sebastes marinus); Salmon (Salmo salar); Sardine (Clupeiformes); Scallop (Pectan magellanicus); Shrimp (Penaeus sp.); Trout, Lake (Salvelinus sp.); and Tuna Fish (Thunnus sp.).

The allergen can be selected from one or more types of animal foods, e.g., Beef (Bus Taurus); Lamb (Ovis aries); and Pork (Sus scrofa).

The allergen can be selected from one or more types of poultry products, e.g., Chicken (Gallus gallus); Egg, Chicken, White (Gallus gallus); Egg, Chicken, Yolk (Gallus gallus); and Turkey (Meleagris gallopavo).

The allergen can be selected from one or more types of dairy products, e.g., Casein, bovine (Bos Taurus) and Milk, bovine (Bos Taurus).

The allergen can be selected from one or more types of nuts, e.g., Brazil Nut (Bertholletia excelsa); Cashew Nut (Anacardium occidental); Coconut (Cocos nucifera); Filbert/Hazelnut (Corylus Americana); Peanut (Arachis hypogaea); Pecan (Carya illinoensis); Walnut, Black (Juglans nigra); and Walnut, English (Juglans regia).

The allergen can be selected from one or more types of miscellaneous materials, e.g., latex, silver, or the like.

The methods for administration of the immunomodulating compositions herein are not limited by the device used to deliver the compositions. Many devices are known in the art that can be used to deliver a known volume of a composition. For example, metered dose devices can be used to administer increasing volumes of a liquid composition so that the amount of allergen delivered to the patient increases with increasing volumes. An ideal device for sublingual or oral delivery consists of a pump system capable of delivering a precise dose of about 0.05 to 1 mL of the therapeutic composition and an actuator with an integrated spray or jet-stream insert. Alternatively, droppers can easily dispense the compositions of the present invention. Exemplary measured dosage devices can also include quick dissolving tablets or films, lozenges, infused papers, graduated measuring cups, or the like. Other routes of administration can employ devices known to those of ordinary skill in the art that are capable of delivering exact volumes of the compositions herein.

In one embodiment, the compositions are packaged in association, along with instructions on the treatment regimen. For example, the instructions can direct a patient to either administer the composition orally, sublingually, or intranasally, and then to repeat the administration at selected intervals, using appropriate doses of the immunomodulating composition (e.g., typically higher doses) with each successive dose until patient sensitivity to the allergen is lowered.

The therapeutic dose for a specific time interval, e.g., a day, a week, or a month, can be included in one or multiple packages or containers as discussed herein. For example, one package can include the initial treatment regimen with a dose for Day 1 and the days thereafter, where the doses are arranged from a lower dose of the allergen to a higher dose, and then a second package with the maintenance treatment regimen for Day 4, 5, or 7, for example, and the days or weeks thereafter, with the same size doses. The doses can advantageously be administered by the patient or a trained clinician, with a minimum onset of oral adverse effects. Preferably, the doses are self-administered without assistance from a medical clinician.

In one embodiment, there is more than one identical dose in the package, e.g., a container of liquid with metered dose dispensing capacity for dispensing equal volumes, or multiple unit-dose containers with the same liquid volume and same allergen concentration. In another embodiment, there are at least two different doses, e.g., multiple unit-dose containers with different liquid volumes and/or different allergen concentrations. The doses are preferably arranged to ensure correct dosing treatment and increase compliance.

The compositions for delivery of antigens, in the form of the allergic component, to a patient can include any dosage form available to those of ordinary skill in the art. Such dosage form can include one or more of: solutions, suspensions, emulsions, creams, tablets, fast-dissolve tablets, capsules, time-release dosage formulations, inhalants, nasal sprays, and nanoparticles. The composition may be provided to the patient in a single concentration of one or more antigens, and the amount of antigen is preferably increased by increasing the number of units of the single concentration provided to the patient. The composition, however, may also be provided in different concentrations.

Oral liquid dosage forms, e.g., solutions, suspensions, and emulsions, are well known to those of ordinary skill in the art. Although the pharmacological effect of a medication is a major factor in symptom management, clinical efficacy is dependent upon several additional factors. This may include the availability of an easy-to-swallow dosage formulation, such as a solution or suspension of an acceptable taste and texture compared to alternatives. As taught herein, the avoidance of phenol represents a uniquely advantageous attribute not previously recognized.

In addition to glycerin, the pharmaceutically acceptable carrier for an oral liquid dosage form typically can also include one or more optional additional additives to advantageously modify one or more formulation properties, including a pH modifying agent (e.g., buffering agent), stabilizing agent, sweetening agent, flavoring agent, colorant agent, preservative agent, emulsifying agent, solubilizing agent, antioxidant agent, or any combination thereof. Any suitable selection or amount available to those of ordinary skill in the art may be included according to the invention so long as the carrier as a whole does not significantly detrimentally affect the stability of the allergen. Significantly detrimentally affecting stability might include reducing stability by more than 50 percent, for example.

A sweetening agent is optionally but preferably included in the carrier. Any suitable sweetening agent available to those of ordinary skill in the art may be used according to the invention. Typically, when present the sweetening agent includes sorbitol, saccharin, acesulfame, e.g., acesulfame potassium, sucralose, xylitol, maltitol, sucrose, aspartame, fructose, neotame, glycerin, sodium saccharate, glycyrrhizin dipotassium, acesulfame K, mannitol, invert sugar, and combinations thereof, or components containing a sweetening agent, such as one or more sucralose-containing components or saccharin-containing components. may be added to modify the taste of the composition. Alternatively, or in addition, a viscous sweetener such as one or more of a sorbitol solution, a syrup (sucrose solution), or high-fructose corn syrup can be used and, in addition to sweetening effects, may also be useful to increase viscosity and to retard sedimentation. A uniquely advantageous attribute is the inclusion of glycerin for the microbial and chemical preservation of the composition, which also provides an acceptable degree of formulation sweetening.

Certain types of sweetening agent may also act as a solubilizing agent or a stabilizing agent, or both, or have other properties, when included as a component of a pharmaceutically acceptable carrier.

An oral liquid immunomodulating dosage form may also contain, in addition to or apart from a sweetening agent, a flavoring agent. Any suitable flavoring agent available to those of ordinary skill in the art may be included in the composition, typically to enhance patient compliance by making the compositions more palatable. The flavoring agent is typically selected in type and amount to increase palatability, e.g., by decreasing or eliminating any undesired taste or off-flavors in the taste, i.e., a taste mask, that would otherwise be detectable by a typical patient to whom the immunomodulating compositions are administered. Examples of a suitable flavoring agent, when used, include one or more natural or artificial flavorings, or both, including but not limited to one or more of menthol, peppermint, anise, and any fruit flavor, such as one or more of grapefruit, orange, banana, lemon, lime, mango, strawberry, pineapple, or cherry, natural and artificial fruit mix flavor, or a combination thereof.

Typical amounts of a flavoring agent, which is optional but preferred, may be present in the carrier in an amount of about 0.05 percent (v/v) to 1.5 percent (v/v), based on the total volume of the composition. Exemplary amounts of flavoring agent can include about 0.2 percent (v/v) to 0.8 percent (v/v) or an amount of about 0.4 percent (v/v) to 0.6 percent (v/v), based on the total volume of the liquid composition.

A colorant agent, when included in the carrier, may be provided in an amount sufficient to provide the compositions with a more aesthetic and/or distinctive appearance. Any suitable colorant agent available to those of ordinary skill in the art may be selected. Typically, a colorant agent suitable for inclusion in the dosage forms includes one or more synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above synthetic organic food additives, etc.), and natural pigments (e.g., beta-carotene, chlorophyll, iron oxide red, etc.). Other suitable colorants include D&C Red No. 33, FD&C Red No. 3, FD&C Red No. 40, D&C Yellow No. 10, and C Yellow No. 6, or any combination of these or the above colorants.

It is optional to include a suitable preservative agent in the carrier, because of the presence of glycerin in certain embodiments. When included, however, most additional preservative agents available to those of ordinary skill in the art may be included, typically in an amount sufficient to extend the shelf-life or storage stability, or both, of the immunomodulating compositions, except that phenol should not be used as a preservative. Preferred examples of a suitable preservative agent, when used, include one or more of: sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenylmercuric nitrate, thimerosal, or any combination thereof.

A preservative agent may be added to the carrier at levels safe for ingestion. Typical amounts of preservative agent, when included, may be from about 0.05 mg/5 mL to 10 mg/5 mL, based on the total volume of the solution. Exemplary amounts of preservative agent can include about 0.3 mg/5 mL to 5 mg/5 mL, based on the total volume of the oral liquid composition.

Emulsifying agents can be used in the carrier an amount sufficient to facilitate more uniform dispersion of an allergen or other excipient, preferably in liquid carriers for components that are not generally soluble in liquid form. Although any suitable emulsifying agent available to those of ordinary skill in the art can be used, if present a preferred emulsifying agent includes gelatin, egg yolk, casein, cholesterol, acacia, tragacanth, chondrus, pectin, methyl cellulose, carbomer, cetostearyl alcohol, cetyl alcohol, or a combination thereof. In one preferred embodiment, the carrier is at least substantially free, or preferably entirely free of egg yolk, alcohol, or both. It may be desirable to minimize or avoid these components because of the potential for allergic reaction in any patient having an allergy to eggs, and/or because of the potential burning or undesirable taste of these types of alcohol components.

Solubilizing agents can optionally but preferably be included, for example, in the carrier in an amount sufficient to facilitate greater or more rapid dissolution of an allergen or other excipient. Preferably, when included, the solubilizing agent is present in an amount sufficient to facilitate dissolving or dispersing the allergen component or other therapeutically active components in the carrier. While any suitable solubilizing agent available to those of ordinary skill in the art can be included in the present formulations, preferably the solubilizing agent may include an alcohol, e.g., 95 percent ethyl alcohol, a glycol, glycerin, D-mannitol, trehalose, benzyl benzoate, trisaminomethane, cholesterol, triethanolamine, sodium carbonate, sodium citrate, sodium salicylate, sodium acetate, and a combination thereof. Preferred alcohols include ethanol, isopropanol, t-butanol, phenol, cresol, a benzyl alcohol, or a combination thereof. Preferably, the solubilizing agent may include a glycol. Suitable glycols may include, for example, those C₂₋₂₀ alkenes functionalized with a glycol, including propylene glycol, polypropylene glycol, polyethylene glycol, etc., or a combination thereof. Preferred glycols include polyethylene glycol, such as PEG-400, or propylene glycol, or both. In one embodiment, however, any solubilizing agent included is typically less than half formed, or at least substantially free, or entirely free, of each of the alcohols listed immediately above, or preferably any alcohol.

Typical amounts of solubilizing agent, when included, are present in an amount of about 1 percent (v/v) to 20 percent (v/v), and more preferably about 4 percent (v/v) to 15 percent (v/v), based on the total volume of the oral liquid composition. Exemplary amounts of solubilizing agent can include about 7 percent (v/v) to 12 percent (v/v) based on the total volume of the oral liquid composition.

A stabilizing agent can include any suitable agent that increases the stability of the allergen. The stabilizing agent can include, for example, one or more liquid excipients such as ethanol, glycerin; one or more glycols, such as polyethylene glycol, e.g., PEG-400, propylene glycol, or polypropylene glycol; a cellulose-based component, such as hydroxypropylmethylcellulose (HPMC) or hydroxymethylcellulose (HMC); or any combination thereof. Thus, it should be understood that certain solubilizing agents may function effectively as a stabilizing agent. For example, propylene glycol may function as both a solubilizing agent and as a stabilizing agent. In one embodiment, as discussed above, the stabilizing agent included is typically less than half formed, or is at least substantially free, or entirely free, of each of the alcohols listed immediately above, or preferably any alcohol.

Examples of a suitable antioxidant, if used, include one or more flavonoids, anthocyanidins, anthocyanins, proanthocyanidins, and combinations thereof. The antioxidant, when used, can help provide long term stability to the liquid compositions, e.g., at ambient conditions for at least about one month, preferably for at least about 3 months, and more preferably for at least about 24 months, or longer, depending on the type and concentration of antioxidant used and depending on other components of the storage microenvironment, such as pH, buffering agent, etc. Even at elevated temperatures, e.g., at least 40° C., the liquid compositions are stable.

Sublingual dosage forms, e.g., rapidly disintegrating tablets or quick dissolving films, are also well known to those of ordinary skill in the art, and typically include one or more of the following: disintegrants, binders, or lubricants, or any combination thereof. Preferred disintegrants include, but are not limited to, starches such as maize starch and rice starch, cross-linked N-vinyl-2-pyrrolidone (CLPVP), sodium starch glycolate, croscarmelose sodium and formaldehyde casein or combinations thereof. A preferred disintegrant is sodium starch glycolate. The disintegrant may be present as an intra-granular disintegrant or extra-granular disintegrant. The proportion of the disintegrant may be about 0.1 to 10 percent, preferably about 1 to 4 percent, and more preferably about 1.5 to 3 percent of the granule or other dosage form.

A binder A binder may be employed, in a minimum quantity to inhibit or prevent unnecessary reduction in the rate of dissolution. A preferred binder is polyvinyl pyrolidone or hydroxymethyl polyvinyl pyrolidone although others such as gelatin may also be used. Preferred binders are soluble in water.

Suitable lubricants for sublingual tablets include, but are not limited to, magnesium or calcium stearates or other long chain fatty acid salts. Magnesium stearate is especially preferred. A minimal proportion of lubricant is preferred, for example 0.1 percent up to about 1 percent, preferably about 0.8 percent. The lubricant may be an intra-granular lubricant, extra-granular lubricant or both. Any use of an extra-granular lubricant alone is preferred to minimize the hydrophobic properties of the dosage form.

The sublingual dosage form, e.g., tablet or film, may also include conventional excipients (except phenols, or in other embodiments, any other alcohols enumerated herein) typically present at up to about 10 percent of the total weight. These may include flavoring agents, for example flavorings such as menthol, peppermint, vanilla or fruit flavorings. Flavoring agents when used are typically present up to about 0.5 to 5 percent by weight of the whole tablet. Sweeteners, such as aspartame or sodium saccharinate may be used, as well as any other sweeteners described herein as suitable for sublingual formulation. Further excipients may also include coloring agents, preservatives and fillers.

Preferred fillers can be selected from, for example without limitation, one or more saccharides. Mannitol, lactose, xylitol and mixtures thereof may be preferred in one embodiment on account of their solubility and despite the water content of lactose in particular. Mannitol may be present in an amount of about 20 to 40 percent, for example about 20 to 30 percent by weight. Lactose may be present in an amount of about 30 to 60 percent, preferably about 45 to 60 percent by weight. In another embodiment, any filler included in the sublingual dosage form is preferably at least substantially free of, or entirely free of mannitol, xylitol, or any combination thereof.

A blister pack is generally used to package sublingual dosage forms, when used. The blister pack may be used for long-term storage, and can include a multi-layered foil laminate having at least one layer of plastic material, preferably as an interior layer, and at least one layer of metallic foil, preferably as a layer opposite the plastic layer from the immunomodulating composition. The laminated structure thus at least includes: a metallic foil layer and a layer of a plastics film, which may itself be a single layer or a laminate. Preferably, the blister pockets within which the sublingual tablets are individually located are formed in the plastic layer. These pockets thus may be formed by injection molding or any other technique available to those of ordinary skill in the art. The plastic layer and/or the metallic layer may have indicia imprinted on a surface thereof. In another arrangement, the blister pack may be in the form of a sandwich structure with metallic foil being laminated to both sides of the plastic layer(s) thereof. In further arrangement, the blister pack may be in the form of a metallic foil having a plastic liner. Two laminated sheets of plastic (independently selected) and metallic foil thus form the pack, with the two plastic layers being adjacent on the inside of the pack and forming the blister or bubble in which the tablets are individually stored. The metallic foil layers thus represent the exterior layers. One preferred metallic foil includes aluminium with polyvinyl chloride (PVC)/thermoelastomer PvdC.

The plastic may be opaque, transparent, or translucent, and may be formed to include a polyester or other plastic material having suitable gas barrier properties. Laminated plastic films, for example containing one or more of PVC, PVdC, EVOH, EVA, polypropylene, LLDPE, and LDPE or the like, or any combination thereof, may be used provided the gas barrier properties of the material inhibit or prevent transmission of air or water vapor therethrough. PVC and TE (thermoplastic) PVdC are preferred in certain embodiments. Where the plastic is laminated, a laminate of PVC and PVdC may be preferred.

A blister pack formed of aluminium foil and PVC/thermoplastic PVdC foil provides an excellent barrier to external contaminants and air, for example. This feature contributes to the long-term storage stability of the sublingual dosage form. The blister pack thus enables storage of the sublingual tablets for an extended period without any significant deterioration of the tablets due to exposure to air or water. The tablets may thus be stored for at least about 2 years, and preferably more than about 3 years, without losing any therapeutic effectiveness or without losing any substantial therapeutic effectiveness.

Intranasal sprays are also well known to those of ordinary skill in the art. These sprays typically include preservatives and tonicity adjusting agents. Common preservatives include one or more quaternary ammonium salts, such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as benzoic acid, sodium benzoate, potassium sorbate, parabens; or complex forming agents such as EDTA; or any combination thereof. The amount of preservatives may range from about 0.001 percent (w/w) to 0.1 percent (w/w). Preferred compositions avoid phenol, and agents such as cetyl pyridium chloride in favor of glycerin, paraben, sodium benzoate, or preservative-free single dose aliquots. Preferred compositions may include about 0.01 percent (w/w) of one or more preservatives. In one embodiment, any preservative included in the intranasal spray is preferably at least substantially free of, benzyl alcohol, chlorobutanol, o-cresol, phenyl ethyl alcohol, or any combination thereof. The use of glycerin in concentrations presented herein provide a uniquely advantageous preservative system requiring no other preservative molecule.

Tonicity adjusting agents, such as sodium chloride, glucose, dextrose, mannitol, sorbitol, lactose and the like may also be added. Their amount is dependent upon the concentration of the other excipients. The tonicity of the composition should approximately be equal to the tonicity of blood. The bulk of the composition is water, preferably demineralised water. Any tonicity adjusting agent, in a preferred embodiment, is typically less than half formed, or at least substantially free, or entirely free, of each of the alcohols listed immediately above, or preferably any alcohol.

An important feature of an oral spray of an intranasal composition is its sprayability, i.e. the ability of the composition to form an aerosol. This ability mainly depends upon the viscosity of the composition. When the composition is too viscous, the composition will not allow the formation of a spray. The composition will form large drops, or the composition may form a jet, when applying the spray device, thus resulting in a high concentration of active ingredient on a small area in the nasal cavity. Such high local concentration usually causes irritation. The composition should therefore have the right viscosity. It should be sprayed widely enough and so the droplets reside long enough in the nasal cavity to allow for sufficient bioavailability. The viscosity of the solution may range up to 50 mPa·s, although those of ordinary skill in the art will be readily able to determine suitable viscosity of the present compositions and for the present methods, particularly based on the guidance herein.

The compositions may adhere to the mucosa, at least to some extent, and this may facilitate retention of the composition of the mucosa and/or enhance the absorption of the allergen(s) in the allergen component. The compositions can be administered via the nasal route using a nasal spray device, pressurized aerosol cannister or simple instillation means. To minimize or avoid overdosing and for hygienic reasons, a unidose nasal spray device is preferred.

The dosage form used to deliver the immunomodulating compositions may be formulated for timed release, e.g. sustained or controlled release of the allergen over a selected period of time. Time-release technology allows for the convenience of administering a single dose, which releases an active ingredient over time, and may keep steadier levels of the ingredient in the blood stream than relying on a clinician or patient to administer additional doses at exact intervals. Sustained release and controlled release dosage forms can be formulated to release an increasing amount of allergen over an extended period of time so as to achieve a constant or step-wise rate of release of the allergen(s), which is particularly useful in the initial treatment phase of the methods herein. The increasing amounts of allergen correspond with therapeutically effective amounts of the allergen component that reduces patient sensitivity to the allergen, and without increase in oral adverse effect, such as oral adverse event frequency. Any of the methods and materials available for formulating time release dosage forms are known by those having ordinary skill in the art and can be applied to the methods and compositions and dosage forms herein, particularly with guidance from the present specification.

It should be understood that in one preferred embodiment, the compositions to be administered include only an allergic component and a pharmaceutically acceptable carrier formed of the glycerin-based diluent, with the remainder being water.

The term “about,” as used herein, should generally be understood to refer to both numbers in a range of numerals. Moreover, all numerical ranges herein should be understood to include each whole integer within the range, e.g., 35 to 65 would include 35, 36, 37, etc. as well as sub-ranges, e.g., 40 to 60, 45 to 55, 42 to 58, etc.

### EXAMPLES

The invention is further defined by reference to the following example, describing in detail of palatability of the present invention. These examples are for illustrative purposes only, and are not to be construed as limiting the appended claims.

### Example 1

### Cat Hair Allergenic Extract With Glycerin Diluent and Fel D 1 Concentration Greater Than US FDA Standardized Cat Hair Extract

A composition of cat hair extract having the following components was prepared.

| | |
|---|---|
| Fel d 1 | >19.9 (20 to 200) FDA AU/mL |
| Glycerin 99.7 percent USP | 52.5 percent (v/v) |
| Sodium Chloride ACS | 0.95 percent (w/v) |
| Sodium Bicarbonate ACS | 0.24 percent (w/v) |
| Sterile Water for Injection USP | q.s. |

Experimental Lot C3921710B prepared by extraction at greater than 1:60 proportion, was more concentrated than FDA limits for Fel d 1 for standardized cat hair (27.5 AU/mL).

### Example 2

### Cat Hair Allergenic Extract With Paraben Preservative

A composition of cat hair extract having the following components was prepared.

| | |
|---|---|
| Fel d 1 | 10-19.9 FDA AU/mL |
| Sodium Chloride ACS | 0.95 percent (w/v) |
| Sodium Bicarbonate ACS | 0.24 percent (w/v) |
| Sodium Bicarbonate ACS | 0.24 percent (w/v) |
| MethylParaben | 0.10 percent (w/v) |
| EthylParaben | 0.10 percent (w/v) |

### Example 3

### Study ALI002-08 a Randomized, Double-Blind, Placebo-Controlled, Parallel Group, Dose-Ranging Study of Sublingual Immunotherapy (SLIT) in Adults Sensitized To the Standardized Allergenic Extract, Cat Hair (Felis domesticus)

In Study ALI002-08, 57 patients were exposed to the undiluted FDA standardized cat hair allergenic extract composition below for 20 weeks.

| | |
|---|---|
| Fel d 1 | 10-19.9 FDA AU/mL |
| Glycerin 99.7 percent USP | 52.5 percent (v/v) |
| Sodium Chloride ACS | 0.95 percent (w/v) |
| Sodium Bicarbonate ACS | 0.24 percent (w/v) |
| Sterile Water for Injection USP | q.s. |

Production Lot # C39032608 met the FDA specifications for concentration of Fel d 1 per mL.

Incidences of oral pruritis or oral paraesthesia were lower than expected and not significantly different between undiluted cat hair extract and placebo. Oral pruritis was experienced by 8.8 percent of those administered the undiluted FDA standardized cat hair allergenic extract compared to 3.4 percent of placebo patients. Oral paraesthesia was experienced by 3,5 percent of those administered the undiluted FDA standardized cat hair extract and 0.2 percent of placebo patients.

Because Esch et al, Ann Allergy Asthma Immunol 2008:100:475-481 had previously reported an incidence of local adverse events, including oral itching, well in excess of 50 percent of patients, even the early unblinded results of study ALI002-08 suggested previously unappreciated differences between the sublingual formulations tested by Esch et. al. and those used in ALI008-02. One difference between those formulations was the incorporation of phenol in the standardized extracts used by the team led by Esch and the specific avoidance of phenol in the formulations prepared by our group. For the first time, it was recognized that the specific formulation of allergenic extracts without phenol might result in important relative formulation advantages.

A new study was designed and conducted to further examine and confirm the previously unrecognized potential advantages of phenol-free versus phenol containing extracts.

### Example 4

### Comparative Assessment of Palatability of Two Sublingual Diluents in Patients

Fifteen men and 15 women with a mean age of 38 years (range, 18 to 61 years) enrolled in this study, with each sampling each formulation in a randomized-crossover format. For the phenol-free glycerin diluent, 87 percent graded the diluent as having a “pleasant” or “very pleasant,” and no patient categorized the solution as unpleasant. For the phenol-containing diluent, 53.3 percent patients gave a grade of “unpleasant and only 6 (20.0 percent) patients gave a grade of “pleasant” The results were overwhelming with 28 (93.3 percent) out of the 30 patients chose diluent A over diluent B as the preferred diluent of choice (p<0.0001).

All thirty patients completed the study and left the clinic without any complaints. As expected the study was not highly sensitive to side effect incidence, with 96.7 percent versus 83.3 percent reporting no adverse event after a single dose test. Only one person described the phenol-free diluent as having a “tingling” while a total of 5 patients described either mouth tingling or nausea as adverse effects following tasting of the phenol diluent. Although there was a greater numerical difference in adverse effects for the phenol-containing diluent, these differences were not statistically significant.

Although conducted by an independent research team, this study was designed by the inventive team to follow on previous surprising evidence that the difference in selected preservative (phenol versus glycerin) could impact the suitability for use by oral or sublingual dosing. With the emergence of sublingual dosing of allergenic extracts, consideration of the taste of alternative diluents could have an effect on the ultimate acceptability of the sublingual formulation of allergenic extracts, however, initial studies of sublingual dosing of pollen extracts have demonstrated no attention to the potential effect on taste and adverse effects of the allergenic extract formulation. Allergenic extracts for injection are formulated with various diluents with 50 percent glycerin and 0.4 percent phenol representing two disparate preservative options.

The objective of the study was to evaluate the palatability and to compare the flavor and taste preferences of two sublingual diluents, one being phenol-free and one containing phenol (Diluent A and Diluent B).

### Subjects

This clinical study was approved by the Medical University of South Carolina Institutional Review Board. The study was conducted at the Medical University of South Carolina. The patients were required to meet the following inclusion and exclusion criteria. Inclusion criteria were: 1) patients with positive allergy testing (either skin-prick test or elevated IgE measured by modified RAST) from 18 to 99 years of age, and able to provide informed consent, perform scale assessment and answer questions related to taste preference; and 2) Patients need to have been fasting for at least 30 minutes prior to study start. Exclusion criteria were: 1) those who have any known food or drug allergies or allergies to the active or inactive ingredients in the test products; and 2) those who had a current medical condition that would interfere with the ability to swallow and/or to discriminate taste (e.g., common cold, sinus or bronchial infection).

Fifteen men and 15 women with a mean age of 38 years (range, 18 to 61 years) enrolled in this study. Of the thirty patients, one was Asian (3.3 percent), 9 (30.0 percent) were African Americans, and 20 (66.7 percent) patients were Caucasians. All but one of the patients were non-smokers.

### Instruments

A modified linear scale previously used in other studies served as a valuable tool in this study. Using this same evaluation tool, a palatability assessment was performed to compare the flavor and taste preference for the two oral diluents in adult patients with positive allergy tests (skin-prick tests or modified RAST) between 18 and 99 years old. The first question was scored from 1 (Very Unpleasant) to 5 (Very Pleasant). The second question asked for preference of either oral Diluent A or oral Diluent B. Any potential side effects were also assessed, such as burning, tingling, nausea, etc.

### Treatments

All subjects received a 3-pumpful dose of one of two solutions, which represented two diluents approved for currently marketed allergenic extracts intended for subcutaneous injection. Use of unchanged subcutaneous allergenic extract formulations for studies of sublingual immunotherapy would represent a rational plan for dosing unless taste or other oral attributes dictate otherwise.

Diluent A: Glycerin—50 percent in water (According to the Invention)

| | |
|---|---|
| Glycerin 99.7 percent USP | 52.5 percent (v/v) |
| Sodium Chloride ACS | 0.95 percent (w/v) |
| Sodium Bicarbonate ACS | 0.24 percent (w/v) |
| Sterile Water for Injection USP | q.s. |

Diluent B: Normal saline with 0.4 percent phenol

| | |
|---|---|
| Phenol | 0.4 percent (w/v) |
| Sodium Chloride | 0.9 percent (w/v) |
| Water for Injection | q.s. |

The order of assignment were randomized in 1:1 ratio between A before B (AB) and B before A (BA) using randomized assignments enclosed in sealed opaque envelopes opened at the time of subject enrollment.

All ingredients listed above are included in the FDA list of approved inactive ingredients and are within the concentrations approved for use. It is noted that phenol is cleared by FDA for use in intramuscular (IM) or intravenous (IV) formulations at concentrations up to 2.5 percent and for subcutaneous (SC) formulations at concentrations up to 0.5 percent. As an inactive ingredient, phenol is not listed in any oral formulations. In oral formulations, phenol is considered an active ingredient, typified by Chloraseptic® spray, which incorporates phenol at 1.5 percent concentrations as an oral anesthetic/analgesic. This concentration is three-fold that proposed in this study, leading to a conclusion of the safety of the proposed phenol concentration. Chloraseptic® spray will not be found in the FDA Electronic Orange Book, Approved Drug Products with Therapeutic Equivalence Evaluations because this product is an old, grandfather-clause product marketed in the US without an NDA.

This was a prospective, randomized, double-blinded pilot study of 30 patients. Patients underwent allergy testing, as part of our standard of care in the adult allergy clinic, and were classified as having allergic rhinitis, i.e., those with positive skin prick test (SPT) or elevated serum IgE as measured by modified RAST. MUSC IRB policies regarding informed consent and HIPAA were followed. Both the physician and the patients only knew the product as Drop A and Drop B. Each patient placed 3 drops (pumpfuls) in the sublingual area beneath the tongue for 2 minutes before swallowing. After each sample, the subjects were provided a 5-point analogue scale to allow ranking of the degree of taste acceptance. After recording the answer, study staff provided the patient with unsalted crackers and bottled water at ambient temperature to cleanse the palate.

Patients were given a 10-minute break between the two tasting periods. If a 10 minute break was not adequate to fully eliminate carryover taste or sensation then the interval was extended up to 1 hour with the time required to clear the aftertaste recorded in minutes. If the time required to clear the aftertaste extends beyond one hour, the subjects were asked to return for the second treatment at least 24 hours later.

After tasting both samples, patients were asked to answer a final question regarding taste preference. A physician or physician assistant was available throughout the study to ensure the safety of the patient. Patients were monitored following the completion of sample tasting for any potential adverse reactions. Any adverse effects occurring during the taste testing session were recorded.

### Results

For Diluent A, 18 (60.0 percent) patients graded the diluent as having a “pleasant” taste, 8 (26.7 percent) patients gave a grade of “very pleasant,” and 4 (13.3 percent) patients said that they were “not sure.” For Diluent B, 16 (53.3 percent) patients gave a grade of “unpleasant.” And only 6 (20.0 percent) patients gave a grade of “pleasant.” Regarding side effects to Diluents A and B, the majority of patients (29 or 96.7 percent vs. 25 or 83.3 percent) did not experience any side effects. Only one person described Diluent A as having a “tingling” on the tongue. For Diluent B, 2 (6.7 percent) patients described Diluent B as “tingling” and 3 (10.0 percent) described as “nausea.” The results were overwhelming with 28 (93.3 percent) out of the 30 patients choosing Diluent A over Diluent B as the preferred diluent of choice (p<0.0001). Finally, of the 28 patients that had preference for Diluent A, all patients stated that the reason for choosing Diluent A over Diluent B was due to the taste of Diluent A. All 30 patients completed the study and left the clinic without any complaints.

The overwhelming preference for Diluent A demonstrates the superiority of sublingual formulations that are at least substantially, or preferably entirely, free of phenol. Diluent A not only performed better in taste tests, but also exhibited fewer adverse side effects. This preference suggests that use of these formulations may improve treatment compliance as well as the outcome of the treatment. Palatability, or taste and smell, is a common deterrent to compliance in children and even in adults, This study confirmed that Diluent A is patient-preferred. Diluent A′s taste makes it easier and more likely for patients to take their medicine as prescribed, and was easier to administer than injection.

Although preferred embodiments of the invention have been described in the foregoing description, it will be understood that the invention is not limited to the specific embodiments disclosed herein but is capable of numerous modifications by one of ordinary skill in the art. It will be understood that the materials used and the chemical or pharmaceutical details may be slightly different or modified from the descriptions herein without departing from the methods and compositions disclosed and taught by the present invention.

What is claimed is:

1. A method for treating a patient suffering from allergy by desensitizing the patient to an allergen component, which method comprises:
orally, sublingually, or intranasally administering to the patient a therapeutically effective amount of an immunomodulating composition comprising an amount of the allergen component sufficient to impart an immune response, and a pharmaceutically acceptable carrier, wherein the carrier is at least substantially free of phenol; and
successively repeating the administration at a selected interval with the therapeutically effective amount of each successive administration comprising an increasing amount of the allergen component, wherein the successive repeating occurs a sufficient number of times to reduce patient sensitivity to the allergen component, and wherein the administering and repeat administering avoids or minimizes oral adverse effects.

2. The method of claim 1, wherein the pharmaceutically acceptable carrier comprises a glycerin-based diluent.

3. The method of claim 2, wherein glycerin is present in an amount of about 35 to 65 percent (v/v) of the carrier.

4. The method of claim 1, wherein the pharmaceutically acceptable carrier further comprises glycerin and at least one of sodium chloride and sodium bicarbonate.

5. The method of claim 4, wherein glycerin is present in an amount of about 25 to 75 percent (v/v), sodium chloride is present in an amount of about 0.1 to 10 percent (w/v), and sodium bicarbonate is present in an amount of about 0.025 to 2.5 percent (w/v), of the carrier.

6. The method of claim 1, wherein the pharmaceutically acceptable carrier further comprises at least one of a saline solution, a buffer, water, or a combination thereof.

7. The method of claim 1, wherein the pharmaceutically acceptable carrier further comprises one or more parabens.

8. The method of claim 7, wherein the one or more parabens is present in an amount of about 0.1 to 1 percent (w/v) of the carrier.

9. The method of claim 1, wherein the immunomodulating composition is at least substantially free of water.

10. The method of claim 1, wherein the allergen component comprises one or more pollens, weeds, epidermals, molds, dust, insects, venoms, or foods, or a combination thereof.

11. The method of claim 10, wherein the allergen component is present in a concentration greater than that present in a standardized extract for the allergen.

12. The method of claim 10, wherein the epidermal is present and comprises one or more types of cat hair, cattle hair, horse hair, mouse hair, rabbit hair, guinea pig hair, hog hair, hamster hair, chicken feathers, duck feathers, or goose feathers, or a combination thereof.

13. The method of claim 12, wherein the cat hair is present in a concentration of about 10,000 BAU/mL to 1000,000 BAU/mL in the immunomodulating composition.

14. The method of claim 1, wherein the administering is with undiluted immunomodulating composition.

15. The method of claim 1, wherein the volume of the immunomodulating composition administered per day is about 0.01 mL to about 1 mL.

16. The method of claim 15, wherein the daily volume is adjusted through control of concentration and drop size to be about 0.05 mL to 0.3 mL.

17. The method of claim 1, wherein the immunomodulating composition is administered in a dosage form comprising a solution, suspension, emulsion, tablet, lozenge, quick dissolving film or quick dissolving tablet, capsule, infused paper or fabric, nasal spray, or combination thereof.

18. The method of claim 17, wherein the dosage form is formulated for timed release.

19. A method for treating a patient suffering from allergy by desensitizing the patient to an allergen component, which method comprises:
providing a plurality of associated therapeutic doses for patient self-administration, wherein the doses of the allergen component are arranged from a lower concentration of the allergen component to a higher concentration of the allergen component and comprise an immunomodulating composition comprising an amount of the allergen component sufficient to impart an immune response and a pharmaceutically acceptable carrier comprising a glycerin-based diluent, wherein the carrier is at least substantially, free of phenol; and
instructing the patient to self-administer the doses orally, sublingually, or intranasally at progressively increasing concentrations of the allergen at subsequent times, wherein the administration has a minimal onset of oral adverse effects.

20. The method of claim 19, wherein the doses are in the form of a solution, suspension, emulsion. tablet, lozenge, quick dissolving film or quick dissolving tablet, capsule, infused paper or fabric, nasal spray, or combination thereof.

21. The method of claim 19, wherein the associated therapeutic doses include more than one identical dose.

22. The method of claim 21, wherein the associated therapeutic doses include at least two different doses of the allergen component.

23. The method of claim 22, wherein the doses are arranged for administration to have quantities of the allergen component that increase by a fixed increment compared to the preceding dose.

24. An immunomodulating product comprising:
a therapeutically effective amount of an immunomodulating composition comprising an amount of the allergen component sufficient to impart an immune response in a patient, and a pharmaceutically acceptable carrier, wherein the carrier is at least substantially free of phenol; and
instructions to:
orally, sublingually, or intranasally administer the composition to the patient; and
successively repeat the administration at a selected interval, with the therapeutically effective amount of each successive administration comprising an increasing amount of the allergen component, wherein the successive repeating occurs a sufficient number of times to reduce patient sensitivity to the allergen component.

25. The product of claim 24, wherein the allergen component is present in a concentration greater than that present in a standardized extract for the allergen.

26. The product of claim 24, wherein the immunomodulating composition is packaged as single dose aliquots.

27. The product of claim 24, wherein the pharmaceutically acceptable carrier comprises a glycerin-based diluent.
